(19) United States
(12) Patent Application Publication
(10) Pub. No.: US 2014/0296706 A1
(43) Pub. Date: Oct. 2, 2014

(54) Devices to Support, Measure and Characterize Luminal Structures

(71) Applicant: InterPerc Technologies, LLC, Atlanta, GA (US)

(72) Inventors: Nicolas Chronos, Atlanta, GA (US); Eberhard Grube, Siegburg (DE); Michael Calhoun, Lighthouse Point, FL (US); Chris Hooper, Atlanta, GA (US)

(21) Appl. No.: 14/352,980

(22) PCT Filed: Oct. 19, 2012
(86) PCT No.: PCT/US2012/061161

§ 371 Date: Apr. 18, 2014

Related U.S. Application Data
(60) Provisional application No. 61/549,058, filed on Oct. 19, 2011.

(51) Int. Cl. A61B 5/107; A61B 5/00; A61B 17/00; A61B 17/34; A61B 6/00; A61B 1/31; A61B 1/313; A61B 17/12; A61B 6/12; A61M 25/09; A61B 19/00

(52) U.S. Cl. CPC A61B 5/1076; A61M 25/09; A61B 5/6853; A61B 5/6858; A61B 5/0004; A61B 5/742; A61B 17/00234; A61B 17/3468; A61B 19/54; A61B 1/31; A61B 1/3132; A61B 17/12136; A61B 17/12168; A61B 6/12; A61B 6/485; A61B 17/12109; A61B 17/1204; A61B 2019/5466; A61B 2017/00243; A61B 2019/5487
USPC 600/424; 600/587; 600/585; 606/130; 606/116; 606/192; 606/198

(57) ABSTRACT

A device for characterizing a luminal dimension, such as the aortic annulus, has also been developed. These include a balloon or basket with sensing and transmitting elements for assessing two or three dimensional shape of lumens using a guide wire and catheter. Sheath introducer devices were developed for percutaneous delivery of bioprosthetic valves during various percutaneous procedures, such as TAVI. A marker needle dispenser for pre-marking anatomical features that are either desirable to target or desirable to avoid has been developed. The needle contains a central passage or lumen for loading marker and spacer material. These are characterized by specific spacing, color, shape or diagnostic imaging criteria to facilitate passage through and placement within the vasculature. Hemostatic stents or balloons are used to prevent bleeding and facilitate closure at sites for entry of catheters or introducer sheaths into luminal structures, especially for procedures such as TAVI through the subclavian artery.

### CROSS REFERENCE TO RELATED APPLICATIONS

This application claims priority to U.S. Ser. No. 61/549,058 filed Oct. 19, 2011.

### FIELD OF THE INVENTION

The invention is generally directed to a sheath introducer for use during endolumenal procedures such as Transcatheter Aortic-Valve Implantation (TAVI), a device for determining an accurate size of an endoluminal structure in which a prosthesis is to be inserted, a device for closure of an endoluminal access entry wound and methods of use thereof

### BACKGROUND OF THE INVENTION

Aortic stenosis, also known as aortic valve stenosis, is a coronary disease characterized by an abnormal narrowing of the aortic valve. The narrowing prevents the valve from opening fully, which obstructs blood flow from the heart into the aorta. As a result, the left ventricle has to work harder to maintain adequate blood flow through the body. If left untreated, aortic stenosis can lead to life-threatening problems including heart failure, irregular heart rhythms, cardiac arrest, and chest pain.

Aortic stenosis is typically due to age-related progressive calcification of the normal trileaflet valve, though other predisposing conditions include congenital heart defects, calcification of a congenital bicuspid aortic valve, and acute rheumatic fever. Conditions including hypertension, diabetes mellitus, hyperlipoproteinemia and uremia may speed up the process. Aortic stenosis is characterized by a long latency period followed by rapid progression after the appearance of symptoms, resulting in a high rate of death (approximately 50% in the first two years after symptoms appear) among untreated patients. Typically, aortic stenosis due to calcification of a bicuspid valve manifests when individuals reach their 40s and 50s, whereas symptoms due to calcification of a normal valve more commonly appear in patients in their 70s and 80s.

For the last fifty years open heart surgery for aortic valve replacement with use of cardiopulmonary bypass, sternotomy (or mini-sternotomy), aortic cross clamping and cardioplegic arrest represents the treatment of choice and the standard of care for patients carrying severe aortic stenosis with symptoms (Bonow, et al., Circulation, 114:e84-231 (2006), Kvidal, et al., J. Am. Coll. Cardiol., 35:747-56 (2000), Otto, Heart, 84:211-8 (2000), Schwarz, et al., Circulation, 66:1105-10 (1982)). However, because the disease most often occurs in the elderly (a prevalence of 4.6% in adults aged 75 years or more), there is a large pool of patients affected by severe aortic stenosis (estimated at 33% of patients with severe symptomatic aortic stenosis) who are not candidates for open heart valve replacement surgery because they are considered too old (nonagenarians, centenaries) for such an invasive procedure, or because they are also affected by other co-existing conditions that compound their operative risk (Jung, et al., Eur Heart J. 26:2714-20 (2005). For these patients, who are at high surgical risk, a less invasive treatment is necessary.

Transcatheter aortic-valve implantation (TAVI) is a procedure in which a bioprostheticbio prosthetic valve is inserted through a percutaneous catheter and implanted within the diseased native aortic valve. The most common implantation routes include the transapical approach (TA) and transfermoral (TF), though trans-subclavian and trans-aortic routes are also being explored (Ferrari, et al., Swiss Med Wkly, 140:w13127 (2010)). These percutaneous routes rely on a needle catheter getting access to a blood vessel, followed by the introduction of a guidewire through the lumen of the needle. It is over this wire that other catheters can be placed into the blood vessel, and implantation of the prosthesis is carried out.

Since 2002 when the procedure was first performed, there has been rapid growth in its use throughout the world for the treatment of severe aortic stenosis in patients who are at high surgical risk, and there is mounting support to adopt the therapy as the standard of care for patients that are not at a high risk for surgery. Clinical studies have shown that the rate of death from any cause at the one-year mark among patients treated with TAVI was approximately 25% (Grube, et al., Circ. Cardiovasc. Interv. 1:167-175 (2008), Himbert et al., J. Am. Coll. Cardiol., 54:303-311 (2009), Webb, et al., Circulation, 119:3009-3016 (2009), Rodes-Cabau, et al., J. Am. Coll. Cardiol., 55:1080-1090 (2010), and the results of two parallel prospective, multicenter, randomized, active-treatment—controlled clinical trials showed that TAVI is superior to standard therapy, when comparing the rate of death from any cause at the 1-year mark (30.7% in the TAVI group, as compared with 50.7% in the standard-therapy group) (Leon, et al., N. Engl. J. Med., 363:1597-1607 (2010)).

Trans-subclavian access is an alternative retrograde pathway that requires a surgical exposure of the left subclavian artery and an adequate minimal vessel inner diameter for 18F delivery systems (6 mm diameter). There are several advantages in using this approach. First, the distance between the site of introduction and the aortic valve is short, compared to the transfemoral option, and it results in a steadier pathway. Second, as long as the subclavian artery is intact, the trans-subclavian procedure can be performed in case of a concomitant vascular disease involving the abdominal aorta or the legs and does not require a thoracotomy. As recently reported by De Carlo, J. A. C. C. (September 2010, abstract TCT-498, in a two year study involving 772 patients, the subclavian approach is a feasible and safe approach, with a higher procedural success rate and lower intraprocedural mortality. Advantages of the procedure as compared to the transfemoral approach are that it bypasses the aortic arch, it is not limited by femoral vasculature, one is able to visually control closure, and there is better control of the delivery catheter and guidewire. Moreover, the axillary-subclavian artery is not usually diseased, there is relatively easy surgical access, and only local anesthetic is required—a major issue with old and very ill patients. Advantages of the procedure as compared to the transapical approach are that it mitigates or eliminates trauma to the heart, can be done under local anesthesia, the recovery time is faster and lower risk, and they do require drainage ports. Drawbacks are the risk of uncontrollable bleeding and a sharp 90° bend to enter the aorta.

Regardless of the implantation route selected, an introduction device (sheath) is used to introduce the prosthetic valve into the site of implantation. The steps for the left subclavian approach including the application of an introducer device are described, for example, in Fraccaro, et al., JACC: Cardiovascular Interventions, 2(9):828-833 (2009). Briefly, according to the Fraccaro procedure, a 5-F sheath is percutaneously placed in the right radial artery through which a 5-F graduate pigtail is advanced in the ascending aorta for hemodynamic monitoring and landmark aortic angiography. A catheter for temporary pacing is advanced through the right cephalic vein in the right ventricle. A cardiac surgeon performs surgical cut-down to isolate the left subclavian artery just below the subclavian bone. A 7-F sheath is then introduced into the subclavian artery, and, using a left Amplatz catheter, a straight 0.035-inch guidewire advanced across the stenotic aortic valve. The direct transvalvular aortic gradient is measured, then, a super-stiff wire is introduced into the left ventricle, the Amplatz catheter removed, and the 7-F sheath replaced with an 18-F 30 cm long sheath. Following balloon aortic valvuloplasty, a CoreValve Revalving System device (porcine pericardial bioprosthesis) is carefully introduced and retrogradely advanced under fluoroscopic guidance over the stiff wire in the ascending aorta across the aortic valvular plane. The valve is then deployed and the delivery system retrieved.

The transition from open heart surgery to catheter-based implantation means that the native valve stays in place and is no longer removed. The selection of the correct plane of the aortic annulus is therefore critical. In addition, exact alignment of the sheath and catheters with the axis of the aorta is imperative for correct implantation.

An important consideration with respect to the introduction device for a prosthetic valve using TAVI is fixation of the introduction device with respect to the apex of the left ventricle. Additional movements of the sheath should be minimized to avoid negatively influencing the correct positioning of the valve. Wisser (Int. Cardiology and Thoracic Surgery, 11:525-526 (2010)) described a method for minimizing movement of the sheath during TAVI, which includes using a rigid table mount instrument holder to which the sheath is fixed in the desired orientation, abolishing any movement. This method requires loosening screws to the mount in the event of an unexpected event. Notwithstanding provisions to minimize movement of the sheath, the TAVI device is still exposed to potential damage as the device transits the sheath.

Another problem with the TAVI procedure is that it is critical to have the correct size device to implant, and this is difficult to determine using existing imaging techniques. The tolerances are very small, and the need for a tight fit essential for a device such as an aortic valve, that it is critical to assess the dimensions of the regions into which the device is to be implanted. If the device is not properly sized and fitted, then leaks may occur.

Additionally, when the TAVI procedure is complete, there is a need for an improved, more precise device and method for closure of the endoluminal entry wound. In the case of subclavian access, there is a degree of difficulty required to isolate and apply adequate hemostasis to the vessel.

Therefore, it is an object of the invention to provide improved introducer devices for percutaneous delivery of bioprosthetic valves during TAVI.

It is a further object of the invention to provide a device which enables the user to more precisely guide the sheath to the desired endoluminal entry site and to apply hemostasis in a controlled, effective manner.

It is another object of the present invention to provide a device which allows for accurate measurements of the site where the prosthetic is to be introduced.

### SUMMARY OF THE INVENTION

Devices and methods for characterizing luminal dimensions, in a preferred example, the aortic annulus and related anatomy, have been developed. In one embodiment, the device is a catheter based, disposable balloon or expandable strut designed basket device for use in characterizing the aorta or aortic annulus. Also provided is a kit for characterizing the aortic annulus prior to placement of an aortic valve. The kit contains the balloon or basket device, usually in combination with a guidewire and a catheter. In yet another embodiment, the device combines both the balloon and the basket designs to provide for the possibility of measuring and dilating, or any combination thereof.

Also provided is a method for characterizing a luminal structure, such as the aortic annulus prior to placement of an aortic valve. The method includes advancing a catheter into the body of a patient, by directing the catheter distal end percutaneously through an entry incision and along a blood vessel until the balloon is located within the annulus.

The devices can also be used in combination, with the needle marker and/or balloon sizing device providing a means to size and assess the shape where the prosthetic is to be implanted, and the sheath introducer insuring that the prosthetic arrives at the implantation site with the correct orientation. These devices may be used in any luminal structure in which there is catheter accessibility and a need for dimensional measurement.

Sheath introducer devices were developed for percutaneous delivery of bioprosthetic valves during TAVI which utilize guidance to place the sheath in the desired endoluminal entry site. The sheath introducer device has applicability for intra-arterial insertion of a wide variety of devices, especially those where there is a degree of criticality in the proper placement of the device within the desired anatomical location. The sheath introducer device includes a feature typically referred to as a referred to as a ‘radiopaque marker’, which guides the sheath as it progresses through tissue.

In some embodiments, the introducer device additionally provides for the protection of ancillary vessels so that the chance for unintended vessel damage is reduced or eliminated. In these embodiments, the introducer is designed to have an atraumatic tip, which can be selectively altered so as to adjust the degree of potential trauma to the surrounding tissue/vessel structures. In addition the tip may be fabricated to allow the physician to steer the device as needed to the desired anatomical location during insertion. Preferably, the tip can be visualized via fluoroscopy so that the physician can position the device as needed to selectively navigate to the desired anatomy. In other embodiments the device is additionally coated to provide the necessary degree of lubricity required to reduce friction, to allow proper placement within the anatomy while minimizing trauma to the wound site where the introducer device is inserted.

In another embodiment, the sheath is designed to accommodate a resectoscope, laparoscope or similar surgical instrument, such that the instrument can be used to optically guide the sheath through the anatomy and visualize the hemostatic device as it is in the target vessel. This embodiment utilizes a hemostatic device which contains a light emitting element capable of emitting light that is visible through the vessel by the resectoscope, laparoscope or similar surgical instrument. The resectoscope, laparoscope or similar surgical instrument can then be used to guide the sheath to the hemostatic device as it appears within the target anatomy. The resectoscope, laparoscope or similar surgical instrument can then be used to visually complete entry into the vessel based on positioning provided by the hemostatic device.

In another embodiment, a marker needle dispenser for pre-marking anatomical features that are either desirable to target or desirable to avoid has been developed. The marker needle dispenser contains a needle, a needle carrier, and a marker dispensing plunger. The needle contains a central passage or lumen for loading marker and spacer material. These are characterized by specific spacing, color, shape or diagnostic imaging criteria to facilitate passage through and placement within the vasculature.

In another embodiment, a hemostatic device is used to help close the site of introduction of the catheter from within the vasculature. This is particularly important in the case of wherein the catheter is introduced into the subclavian artery and from there into the heart. This artery is accessed in an area surrounded by nerves and the clavicle and is particularly difficult to immobilize and close. The hemostatic device is introduced via the brachial artery and slid into position as the catheter is withdrawn. It has an element capable of emitting an optically detectable visual light such that the light is transmitted through the luminal structure (in the case of a TAVI procedure, a blood vessel) and can be viewed with a resectoscope, laparoscope or similar surgical instrument. The light emitting portion of the hemostasis device serves to produce an optically detectable marker for identification of the preferred location to access the luminal structure. The hemostasis device also contains a radiopaque marker(s) to aid in placement of the device under fluoroscopy. These marker(s) also aid in the positioning of needle delivered radiopaque markers to externally denote where the entry site is located. These external luminal markers are utilized under fluoroscopy to close the wound site at the completion of the case. It may be used to immobilize the artery during closure, to position a hemostatic stent, or to apply pressure at the site during closure and shortly thereafter until the risk of bleeding is diminished.

### BRIEF DESCRIPTION OF THE DRAWINGS

FIG. 1 is a schematic of the heart showing the valves and chambers and vasculature.

FIGS. 2A and 2B are cross-sectional views showing the insertion of the guidewire and catheter into the subclavian artery (FIG. 2A) or the femoral artery (FIG. 2B) and from there into the heart for the purpose of TAVI placement.

FIG. 3 is a cross-sectional schematic showing the inserted prosthetic heart valve in place within the aorta.

FIG. 4 is a schematic of a system, depicting placement of a device for characterizing the aortic annulus.

FIGS. 5A, 5B and 5C are prospective views of an expandable basket type device for characterizing the aortic annulus. FIG. 5A is the expanded device and FIG. 5B shows the collapsed device for transport to the annulus. FIG. 5C is another embodiment of the basket design device.

FIGS. 5D and 5E are perspective views of a basket device containing within the basket structure, a valve mechanism capable of stopping fluid flow by opening or closing and allowing the flow of fluid, such as blood. FIG. 5E depicts the valve inside a basket device in a non-deployed position.

FIGS. 6A and 6B are perspective views of the basket device shown in FIG. 5A combined with a balloon device, so that dilatation is possible either before or after luminal measuring has been performed.

FIG. 7A is a perspective view of the expanded device in the measuring mode within the annulus; FIG. 7B is a perspective view of the collapsed device for transport to or from the annulus.

FIG. 8A is a schematic of a sheath showing the external structure of the introducer sheath into which the catheter for delivery of a device is inserted. FIG. 8B shows alternative designs for the tip of the introducer sheath.

FIGS. 9A and 9B are schematics showing the marker needle dispenser. FIG. 9A shows the marker needle containing the market elements. FIG. 9B shows the deployed markers relative to the needle dispenser.

FIG. 10 is a prospective schematic showing the insertion of the hemostasis catheter into an artery and positioning of the device such that the distal radiopaque marker elements are positioned to aid in placement of the catheter sheath at a different arterial location.

FIG. 11 is a cross-sectional schematic of a catheter and introducer sheath inserted into a hemostatic device positioned adjacent to markers inserted using the marker needle.

### DETAILED DESCRIPTION OF THE INVENTION

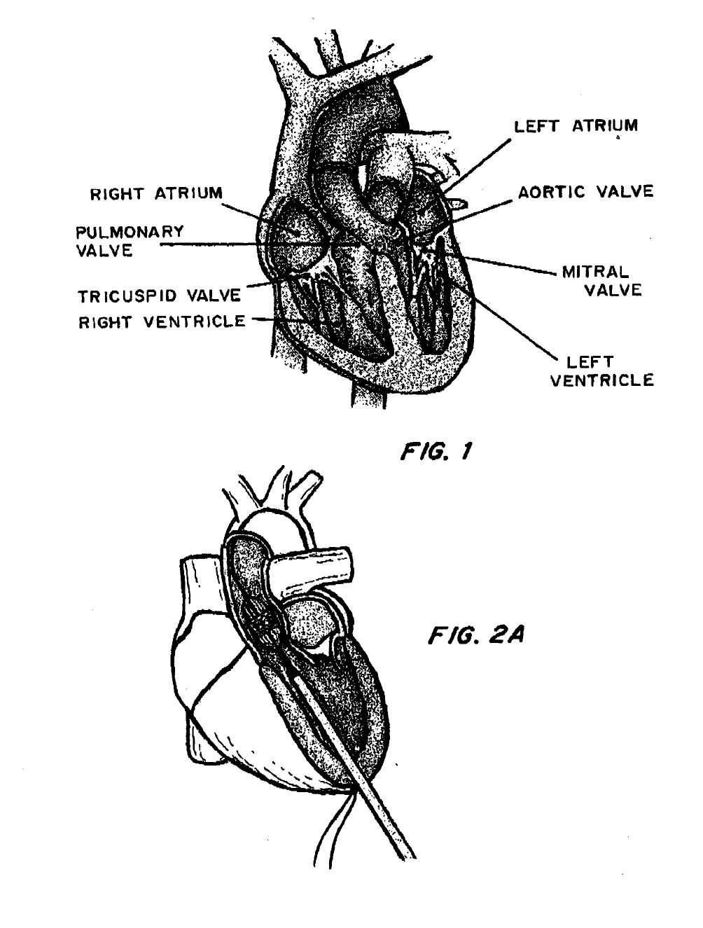

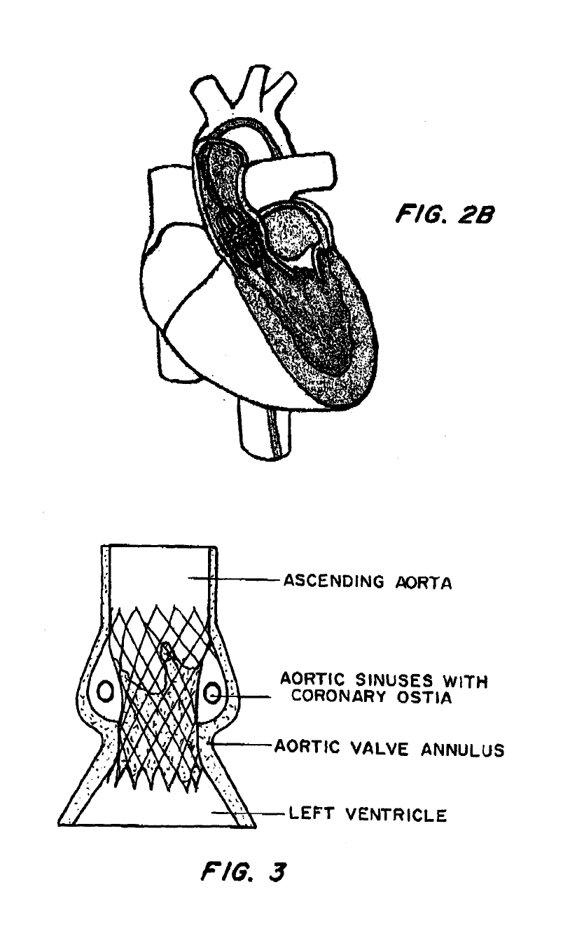

FIG. 1 is a cross-sectional view of the heart. In procedures such as TAVI, a catheter is inserted through an introducer sheath into the chambers of the heart to position a replacement aortic valve over and in place of the existing valve. Different approaches may be used to achieve the same goal, as shown by FIGS. 2A (transapical approach) and 2B (transfemoral approach). FIG. 3 shows the inserted device, positioned between the ascending aorta and the left ventricle, at the annulus.

Commercially available values include the Medtronic CoreValve® and the Edwards Sapien™ THV. The CoreValve® system is a self-expandable nitinol stent with an inner porcine pericardial valve, designed to sit into the aortic root and to anchor into the aortic annulus. However, the valve function is more supra-annular and a skirt of pericardium, bordering the lower portion of the stent-valve, prevents paravalvular leaks. The valve is available in two sizes, the 26 mm and the 29 mm, and the delivery system (only for retrograde applications) has an external diameter of 18F allowing for a transfemoral (TF) (recommended) or a trans-subclavian (off-label use) transcatheter stent-valve implantation. The Edwards Sapien™ THV stent-valve is a tubelike stainless steel balloon-expandable stent, of 14-16 mm in length, with an inner bovine pericardial valve treated with the ThermaFix™ anticalcification system for transfemoral and transapical (TA) applications. The stent-valve is inserted and deployed within the aortic annulus and sits in a sub-coronary position. The Sapien™ stent-valve is available in two sizes, a 23 mm (14 mm length) and a 26 mm (16 mm length), and can be introduced via a transapical (antegrade, Edwards Ascendra™ system) or a transfemoral (retrograde, Edwards RetroFlex™ system) transcatheter approach that requires a 22F (for the 23 mm) or a 24F (for the 26 mm) sheath.

As is readily apparent in this particular application, it is critical that the correct size valve be used to replace the existing valve, so that it fits securely and does not leak once implanted, and that this device be as small as possible to minimize complications associated with transport through the blood vessels into the heart. As is also apparent, the heart and blood vessels exhibit wide variability among individuals, especially those with heart disease, and it is extremely difficult to use current forms of imaging to see within the vessels and the heart to form a two or three dimensional rather than two dimensional image at the site of implantation.

The variation in dimensions, angles, diversions, and other anatomical variations, as well as normal bends, narrowing and turns of the vasculature, increase the difficulty in properly aligning a device using a catheter, even with a guidewire, which can twist during introduction, resulting in the device not being properly aligned when it reaches the site of implantation and can result in trauma to the vessel wall during introduction, causing damage to the epithelial lining, swelling or performation.

### I. Catheter Based Device for Characterizing Luminal Dimensions

Devices are provided for characterizing luminal dimensions such as the aortic annulus and related anatomy. These are particularly useful in conjunction with the TAVI procedure, where it is necessary to accurately determine the size of the aortic annulus in order to properly size a percutaneously delivered TAVI device. The system and device can be used for characterizing other lumenal structures, however, particularly in the case of assessing lumens which are not round and therefore where analysis using current two-dimensional techniques lacks precision. These include measurements of the vena cava, mitral and cuspid annulus, and esophageal dimensions, as well as other luminal structures.

A. Balloon and Basket Devices

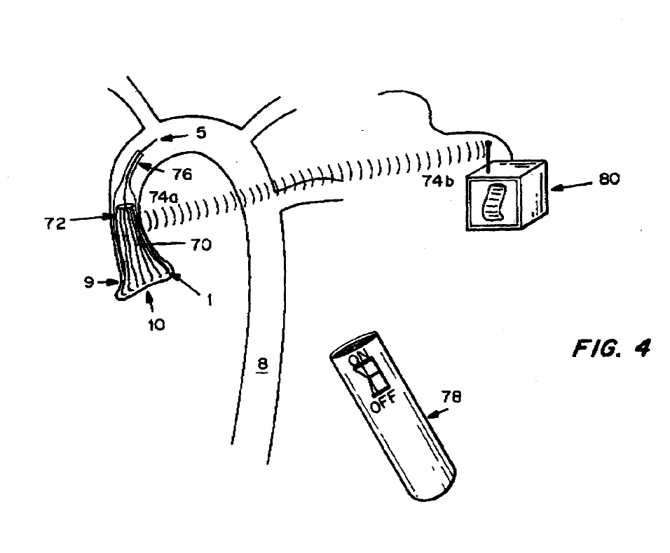

A system for providing a two and/or three-dimensional characterization of lumens has been developed. As depicted in FIG. 4, the device includes a balloon 70 (or in an alternative embodiment, a basket) that is deployed at the site to be characterized, and includes multiple sensors or filaments 72 that can detect points of impact within the lumen and a transmitter 74A that sends signals remotely, preferably wirelessly, to a remote processer 74B. A guidewire 75 is used to position the balloon 70 within the catheter body 76. Using a remote control 78, the physician is able to take measurements as necessary. The display 80 depicts an image of the location where the balloon 70 is inflated. The measurements are transmitted to a receiving and/or display device 80, where the image is reconstructed from the measurements. In this embodiment, the elements 72 are depicted as electrical, however they may also be fiberoptic or a combination of fiber optic and electrical. In this embodiment, the data transmission includes wireless transmission 74A and receiving 74B elements on the distal portion of the catheter 76 and on the data collection device 80, respectively. In another embodiment, there is a hard wire connection from the catheter to the data collection device 80. In one embodiment of the control device for data acquisition, there is a foot pedal, a touch screen control or various other commonly used interfaces, such as a remote control handle with on/off switch 78.

Balloon Device

The balloon design provides for minimally interrupted blood to flow through a lumen or other facsimile during balloon inflation/data gathering. The balloon is preferably a hydraulically actuated, low pressure, conformal design that may have one or more layers. The layer contacting the aorta is intended to provide a conformal fit such that when inflated, the balloon defines a reverse image of the aorta. This portion of the device is not intended for displacement of aortic tissue. The preferred approach for use of this device is from the Femoral artery, however, the subclavian approach may be used.

In another embodiment, the balloon is designed to stop the flow of fluid when inflated and serves to act as a closed valve during inflation.

The balloon can be composed of any suitable material, for example, nylon or nylon copolymer (compliant, non-puncture) or polyethylene terepthalate (“PET”) (high pressure, non-compliant) with a urethane, polymer, or other material. The balloon may be a multi-layered balloon with a non-compliant inner layer to a most compliant outer layer or multilayered with similar material. For example, an inner most layer of PET, which provides a higher pressure balloon, may be surrounded by an outer layer of nylon, which provides a more puncture-resistant surface. The device may have a dilatation balloon within the measurement balloon (or the reverse).

The diameter and length of the balloon is selected based on the diameter of the patient's vasculature, the size and shape of the bend in the guidewire, and/or the location or locations where the protective buffer is deployed. The guidewire fits into a through lumen of the balloon and the balloon is slideable about the guidewire. For example, the balloon diameter can range from 17 mm to 30 mm with a preferred diameter of 20 mm to 30 mm and the balloon length can range from 1 cm to 3 cms, with a preferred length of 1.5 cms.

The balloon may be coated with technology to provide for lubricity, radiopacity, electrical isolation, biocompatibility and/or other coatings known to one of ordinary skill in the art. These stents and/or balloons may also include an optical or non-optical element as part of the catheter construction to aid in marking device position for proper anatomical location of the catheter.

The balloon is preferably a hydraulically actuated, low pressure, conformal design that may have one or more layers. The layer contacting the aorta is intended to provide a conformal fit such that when inflated, the balloon defines a reverse image of the aorta. This portion of the device is not intended for displacement of aortic or vascular tissue. In other embodiments the device has a dilatation balloon within the measurement balloon (or the reverse) such that the physician could measure, dilate and measure post dilatation the aorta or aortic annulus prior to TAVI placement.

Basket

The basket term is used to generally describe the device appearance. This device may also be described as spreadable data gathering elements, a snare, or many other descriptors. The basket provides for minimally interrupted blood to flow through the device during data gathering. The basket is preferably a mechanically actuated, controlled pressure, conformal design that may have one or more elements. The portion contacting the aorta is intended to provide a conformal fit such that when deployed, the basket defines a reverse image of the aorta. This portion of the device is not intended for displacement of aortic tissue. The preferred approach for use of this device is from the Femoral artery, but may also be used in other approaches such as brachial or subclavian.

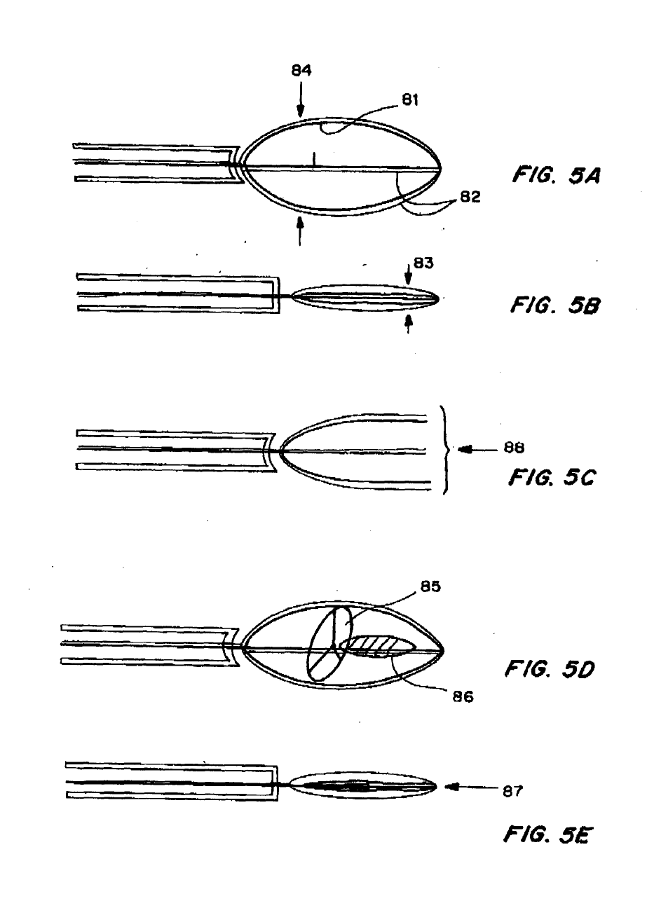

In an embodiment shown in FIGS. 5A, 5B and 5C, the device 84 is a catheter based basket design in which the basket contains both structural elements 81 as well as measuring elements 82. These elements 81 and 82 are placed within the target anatomy in a retracted position 83 (FIG. 5B) and are deployed 84 (FIG. 5A) once the preferred site has been reached. Once deployed, these elements are capable of taking an accurate two or three dimensional representation of the target anatomy. FIG. 5C depicts another embodiment 88 of the basket design device.

The basket can be composed of any suitable material, for example, stainless steel, nitinol or polymers as commonly used in fiber optic construction. The basket may form any of the shapes (but not limited to) as seen in FIGS. 84 and 89. The basket may be a multi-layered device, one example would have an inner layer of elements designed for the purpose of deployment and support, combined with an outer layer of elements designed to gather dimensional data. It would also be possible to reverse this arrangement such that the structural and data gathering elements were reversed from the initial example

The diameter and length of the basket is selected based on the diameter of the patient's vasculature, the size and shape of the bend in the guidewire, and/or the location or locations where the protective buffer is deployed. The guidewire fits into a through lumen of the basket and the basket is slideable about the guidewire. For example, the basket diameter can range from 17 mm to 30 mm with a preferred diameter of 20 mm to 30 mm and the basket length can range from 1 cm to 3 cms, with a preferred length of 1.5 cms.

The basket may be coated with technology to provide for lubricity, radiopacity, electrical isolation, biocompatibility and/or other coatings known to one of ordinary skill in the art.

In another embodiment shown in FIGS. 5C and 5D, the basket device 90 contains, within the basket structure 81, a valve mechanism capable of stopping fluid flow by opening 85, or closing 86 and allowing the flow of fluid, such as blood. FIG. 5D depicts the valve 86 inside a basket device 92 in a non-deployed position. The basket valve mechanism is capable of being selectively opened and/or closed such that fluid flow through the catheter can be allowed or prevented.

Combination Basket-Balloon Device

The basket and balloon devices may be combined into one device.

The balloon may be contained within the same axial space as the basket, or may be positioned at a different axial location.

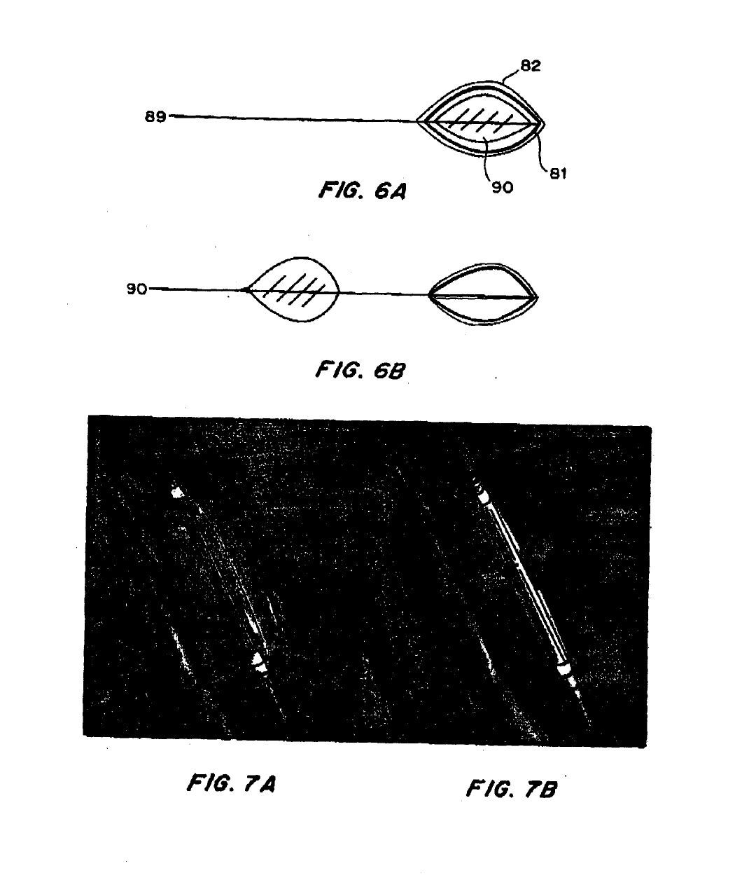

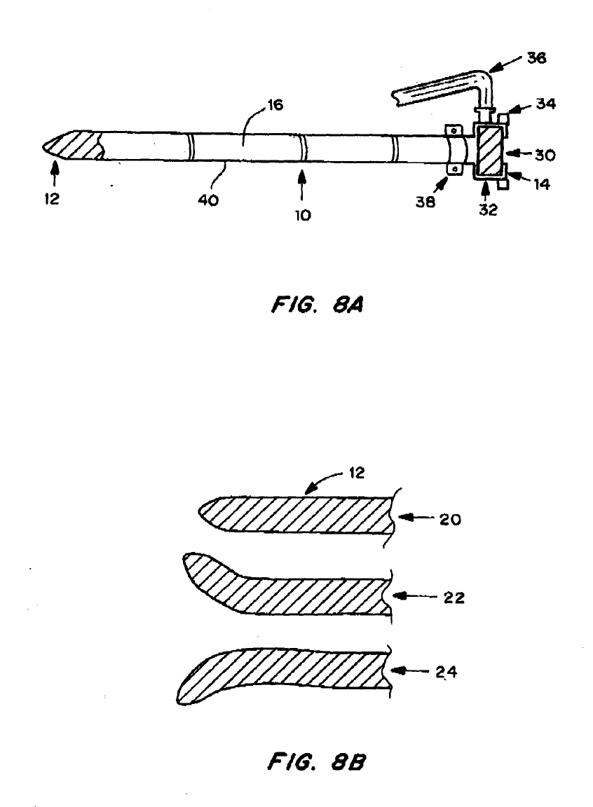

In the embodiment shown in FIGS. 6A and 6B, the basket device shown in FIG. 5A is combined with a balloon device 90, so that dilatation is possible either before or after luminal measuring has been performed. The balloon device 90 may be located within the basket structure 81, as depicted in FIG. 6A as device 89, or placed at a different axial location from the basket structure 81, as shown in FIG. 6B as device 90. This embodiment may be possible with or without a valve mechanism.

Also provided is a kit for characterizing a luminal structure, such as the aortic annulus prior to placement of a TAVI. The kit contains the balloon or basket device, usually in combination with a guidewire and a catheter.

Lubricity

It may be desirable to coat the surface of the balloon or the basket elements so as to avoid potential tissue irritation resulting from abrasion and puncture during handling and use. Methods for coating devices are known in the art. A variety of urethane based coating compositions for medical applications are known in the art. For example, U.S. Pat. No. 4,642,267 to Creasy et al. describes hydrophilic polymer blends (a thermoplastic polyurethane and a hydrophilic poly (N vinyl lactam) such as polyvinylpyrrolidone) useful for coating catheters and other surfaces. Additional components such as crosslinking agents and wax dispersions can be added to the blend. U.S. Pat. No. 4,675,361 to Ward, Jr. relates to polymer systems useful for coating surfaces having blood and tissue contacting applications. U.S. Pat. No. 5,272,012 to Opolski describes a coating solution which contains a protective compound such as a urethane, a slip additive such as a siloxane, and optionally, a crosslinking agent for the protective compound such as a polyfunctional aziridine, coating the solution onto a surface of a medical apparatus and allowing the coating to set.

Radiopaque Materials

Radiopaque materials suitable for use with the devices disclosed herein include, but are not limited to barium, bismuth, tungsten, iridium, iodine, gold, iron, and platinum. A single radiopaque material may be used or such materials may be mixed in various ratios to provide the desired radiopacity characteristics. As is appreciated by one of skill in the art, different radiopaque materials may be disposed on/in different regions of the balloon in various combinations to achieve the desired radiopacity. The radiopaque material may be incorporated into various layers through admixing the material with, for example, a polymeric coating material. However, the radiopaque material may also be applied by any other method known in the art. Such methods include, but are not limited to coatings, electroplating, chemical vapor deposition (CVD), physical vapor deposition (PVD), and ion beam assisted deposition (IBAD). One or more methods may be employed depending on the desired characteristics of the radiopaque layer, such as thickness, flexibility, radiopacity and the like. Additionally, one layer of radiopaque material may be directly applied to the surface of another. Typically, radiopaque materials are combined with a polymeric coating material and coated onto one or more layers of the balloon. As such, the radiopaque material may be coated onto a layer of the balloon by spray coating, dip coating, dispense coating, printing or any other method commonly known to one skilled in the art.

Dilation Balloon

In some embodiments the device has a dilatation balloon within the measurement balloon (or the reverse) such that the physician could measure, dilate and measure the annulus size post dilatation prior to TAVI placement. Dilatation may be advantageous in the case of an annulus that has a constriction and as such does not fit within one of the commonly available TAVI device sizes. Typically available TAVI device sizes are 23 mm, 26 mm, 29 mm and 31 mm in diameter. The combination of a dilatation device and an annulus measurement device benefits the patient and the physician by limiting the need to perform device exchanges within the anatomy along with a reduction of the related complications. This combination allows the physician to size the annulus, modify/optimize the diameter through use of a dilatation balloon and place a TAVI device without the need to perform several device exchanges

B. Data Gathering Elements

Balloon Elements

The balloon contains elements which are capable of taking data, in the example of a TAVI procedure, from the aorta, the aortic annulus and other landmarks, including the coronary ostium, such that a two or three dimensional reconstruction image of the aortic area can be reproduced. These data gathering elements may be constructed from fiber optic materials, electrically conductive materials or a combination thereof. These data gathering elements may be able to detect and dimensionally measure aortic tissue as well as may detect and measure the other data gathering elements. These measurements may also utilize a common reference point. This internal element recognition feature serves to provide a robust dimensionally and anatomically accurate reconstructed image of the aortic area.

The elements may be fixed or printed directly on the inside of the primary measurement balloon material, or may be fabricated separately and then fixed to the inside of the primary measurement balloon. These elements may also be fixed or printed on the outside of a balloon. These elements will either be constructed of materials with known biocompatibility or physically isolated such that biocompatibility is provided during use or some combination thereof. In the case of electrically isolated elements, isolation can be accomplished by use of biocompatible materials such as Parylene, Silicone, Polytetrafluoroethylene (PTFE) or other similar biocompatible dielectric materials known in the art.

In the case of electrically active balloon measurement elements, these elements are electrically active to the point needed for data acquisition and transmission, but utilize safe design and voltage levels for use within the coronary anatomy as typical of current medical technology. These elements may be powered by battery, live electricity or a combination.

In the case of fiber optic balloon measurement elements, these elements utilize light for activation, but utilize safe design and temperature levels for use within the coronary anatomy as typical of current medical technology. These elements may be powered by battery, live electricity or a combination.

In the case of a combination of electrical and fiber optic elements, similar design parameters are employed as with the electrical and fiberoptic designs previously mentioned.

In the case of use of any combination of these elements, biocompatibility will be provided by use of material and methods know in the art.

Basket Elements

The basket contains elements which are capable of taking data from the aorta, the aortic annulus and other landmarks, including the coronary ostium, such that a two or three dimensional reconstruction image of the aortic area can be reproduced. These data gathering elements may be constructed from fiber optic materials, electrically conductive materials or a combination thereof. These data gathering elements are able to detect and dimensionally measure aortic tissue as well as detect and measure the other data gathering elements. These measurements also utilize a common reference point. This internal element recognition feature serves to provide a robust dimensionally and anatomically accurate reconstructed image of the aortic area. These elements form an array that may be considered static such that once in position, physical movement is not required in order to take an image. In another embodiment, dynamic movement of these elements may be necessary in order to take a measurement.

The elements may be printed directly on the inside of the primary measurement basket material, or may be fabricated separately and then fixed to the inside of the primary measurement basket. These elements will either be constructed of materials with known biocompatibility or physically isolated such that biocompatibility is provided during use or some combination thereof. In the case of electrically isolated elements, isolation can be accomplished by use of biocompatible materials such as Parylene, Silicone, Polytetrafluoroethylene (PTFE) or other similar biocompatible dielectric materials known in the art.

In the case of electrically active basket measurement elements, these elements are electrically active to the point needed for data acquisition and transmission, but utilize safe design and voltage levels for use within the coronary anatomy as typical of current medical technology. These elements may be powered by battery, live electricity or a combination.

In the case of fiber optic basket measurement elements, these elements utilize light for activation, but utilize safe design and temperature levels for use within the coronary anatomy as typical of current medical technology. These elements may be powered by battery, live electricity or a combination.

In the case of a combination of electrical and fiber optic elements, similar design parameters are employed as with the electrical and fiberoptic designs previously mentioned.

The elements may be activated on demand by the physician by use of or combination of a hand control on the proximal portion of the catheter assembly, by a foot pedal or by wireless derivatives of each, although activation methodology is not limited to these examples. There may also be a provision to allow for someone other than the physician, either in or out of the surgical field or remotely, to collect the data. The physician may place the measuring device in a patient, while a person not in the surgical field, or even someone at a remote location may be able to operate the data acquisition portion of the device. This is enabled by software allowing the device to be connected to a network such that a person on the network has access and the ability to obtain and analyze data taken from the device. The basket measurement elements may carry data from the distal portion of the catheter to the proximal portion through a hard connection to a collection source These data collection sources may range from, but are not limited to, a computer network, smart phone, desk top computer, computer tablet or other commonly used forms of technology for storing, manipulating and displaying data. There may also be a wireless provision to enable data to be transmitted directly from the catheter to the collection/display source. This wireless transmission of data may be accomplished by use of typical wireless protocols such as Bluetooth, radio frequency, microwave, infrared or other common wireless data transmission methods. It should be noted that any of these methods may require modification for use within an Operating Room/Hospital environment such that interference with other equipment is prevented. There may also be a provision within the collection/display source to collect the data for historical purposes such as post procedure evaluation or inclusion into a patient's medical record.

C. Catheter and Guide Wire

Balloon catheters are used in a variety of therapeutic applications, including intravascular catheters for procedures such as angioplasty treating coronary, neurological and peripheral blood vessels partially or totally blocked or narrowed by a stenosis. Any of the balloon catheters known in the art for cardiac application can be used to deploy the balloon device described herein. Most balloon catheters have a relatively long and flexible tubular shaft defining one or more passages or lumens, and an inflatable balloon attached near one end of the shaft. This end of the catheter where the balloon is located is customarily referred to as the “distal” end, while the other end is called the “proximal” end. The balloon is connected to one of the lumens extending through the shaft to selectively inflate and deflate the balloon. Examples of balloon catheters are shown in U.S. Pat. No. 5,304,197, to Pinchuk et al. and also in U.S. Pat. No. 5,370,615, to Johnson.

Basket catheters are used in a variety of therapeutic applications, including capture and removal of thrombus. Any of the basket catheters known in the art for cardiac application can be used to deploy the basket device described herein. Most basket catheters have a relatively long and flexible tubular shaft defining one or more passages or lumens. The end of the catheter positioned within the patient is customarily referred to as the “distal” end, while the other end is called the “proximal” end. The basket is connected to one of the lumens extending through the shaft and then coupled to a mechanism to allow for the physician to selectively open and close the basket. Examples of basket catheters are shown in U.S. Pat. No. 6,748,255, to Fuimanon et al. and also in U.S. Pat. No. 5,997,536, to Osswald.

In some embodiments, the catheter contains a hard connection to a collection source, which carries data from the basket at the distal portion of the catheter to the proximal portion. Typically the catheter may have a wired connection directly into a collection source. These connections are typically constructed of an element that plugs into, or otherwise provides a method for stable connection of the catheter to the data collection source such that data can be reliably transferred from the catheter to the data collection source. This can be accomplished by use of a plug, a pin connector, a twist connector or other methodologies commonly used in the telecommunications industry for the connection of data transmission elements. In the event wireless data transmission is desired, an element designed to send/receive wireless data is constructed such that it is a biocompatible, integral feature of the proximal end of the catheter. A similar device is connected either directly to the data connection source, or to a node capable of sending/receiving data to the remote data collection source. These data collection sources may range from, but are not limited to, a computer network, smart phone, desk top computer, computer tablet or other commonly used forms of storing and manipulating data.

The measuring devices can be deployed using into any of the conventional guidewires available in the art. For example, guidewires such as those sold by Medtronic, Inc, Vascular Solutions, Inc., Boston Scientific and Abbott Vascular. A suitable guidewire is selected based on a number of factors that known in the art, including, but not limited to, 1) compatibility with the patient (i.e. luminal diameter of the patient), 2) the device to be delivered, and 3) the route of delivery, for example, the brachial artery.

In preferred embodiments, the guidewire is between about 0.030 inches to 0.038 inches in diameter with a preferred diameter of 0.038 inches.

In the most preferred embodiment, the guidewire has a stainless steel core and a polytetrafluoroethylene (PTFE) coating. An exemplary guidewire is an Amplatz Super Stiff™ Guidewire available through Boston Scientific

Other guidewires useful for feeding a catheter through a body duct to a distant target site are described, for example, in U.S. Pat. No. 5,902,254 to Magram, U.S. Pat. No. 6,039,743 to Quiachon, et al., U.S. Pat. No. 6,033,720 to Stolze, et al., and U.S. Pat. No. 6,500,130 to Kinsella, et al. Catheters for insertion into a body vessel are described for example in U.S. Pat. No. 6,866,655 to Hackett. Suitable catheters and guidewire assemblies are described in U.S. Pat. No. 7,914,466 to Davis, et al.

In the most preferred embodiment, the guidewire has a stainless steel core and a polytetrafluoroethylene (PTFE) coating. An exemplary guidewire is an Amplatz Super Stiff™ Guidewire available through Boston Scientific.

Sterile disposable kits for measuring the aortic annulus that properly sized TAVI devices can be selected and placed are provided. The kit contains an annulus measuring device, as described above. The kit can further contain a guidewire and/or a catheter. The inflatable devices can be deployed using into any of the conventional guidewires, such as those sold by Medtronic, Inc, Vascular Solutions, Inc., Boston Scientific and Abbott Vascular.

The guidewire and catheter are used to advance the balloon into the body of a patient, by directing the catheter distal end percutaneously through an incision and along a blood vessel until the balloon is located within the annulus. As shown in FIGS. 7A and 7B, the balloon is positioned directly into the aorta such that, prior to inflation, the distal portion of the balloon approximates the position of the aortic annulus. The balloon is inflated to approximate the interior of the aorta, including the aortic annulus, while allowing for blood flow to continue through the balloon. Proper location of the balloon is confirmed through use of radiopaque markers located on the balloon and use of fluoroscopy with contrast media to define the vasculature as is typical practice.

The device will obtain measurement data of the aortic anatomy by use of a low pressure conformal balloon. As shown in FIGS. 5A-5E, the balloon will contain several elements disposed within the wall of the balloon such that these elements will come in contact either directly, or indirectly, with the aortic tissue. These elements contain features that allow them to report a position within the aorta to a central reference point and in some embodiment, from and/or to each of the other elements. After the measurements are taken, the balloon is deflated and withdrawn, as shown in FIG. 7B.

D. Data Collection

There is a collection source outside of the patient field. This collection source may be generically referred to as a ‘box’. This ‘box’ is reusable and designed to capture, process and prepare data for display so that the physician can easily utilize it for sizing a TAVI device. The collection source may display the data, or may send the data to another device for display, such as a monitor, computer, tablet or smart phone. These collection sources will function in a similar manner as a computer in that data is acquired by the catheter, sent to the collection source, analyzed and a usable result displayed such that the user may determine dimensional information related to the coronary anatomy. The collection source will contain software that takes the raw data, performs analysis and from that analysis, reconstructs an image of the vascular anatomy such that dimensions can be taken from the reconstruction for use in optimizing the size of intravascular devices.

In some embodiments, the catheter contains a hard connection to a collection source, which carries data from the balloon at the distal portion of the catheter to the proximal portion. Typically the catheter may have a wired connection directly into a collection source. These connections are typically constructed of an element that plugs into, or otherwise provides a method for stable connection of the catheter to the data collection source such that data can be reliably transferred from the catheter to the data collection source. This can be accomplished by use of a plug, a pin connector, a twist connector or other methodologies commonly used in the telecommunications industry for the connection of data transmission elements. In the event wireless data transmission is desired, an element designed to send/receive wireless data is constructed such that it is a biocompatible, integral feature of the proximal end of the catheter. A similar device is connected either directly to the data connection source, or to a node capable of sending/receiving data to the remote data collection source. These data collection sources may range from, but are not limited to, a computer network, smart phone, desk top computer, computer tablet or other commonly used forms of storing and manipulating data.

This information is used to construct a two or three dimensional recreation of the aorta via software residing in a data collection device. This software will interpret the position of each individual element relative to a central reference point along with the position of each element relative to the other elements such that construction of a two or three dimensional aortic representation is possible. In another embodiment, each data element will be able to determine its own particular position relative to itself and data from all elements will be combined via a software algorithm to construct a two or three dimensional aortic representation if possible. Once the construct has been rendered, the software will allow for the selection of various anatomical features, including, but not limited to, the aortic annulus. The software will be capable of rendering a two or three dimensional representation of the aorta on a data collection screen and will enable the user to take necessary measurements directly from this graphical representation. The measurements derived from the use of the software will correlate directly to common nomenclature and sizes of TAVI devices. The software will also have a capability to determine the annulus measurement and provide the user that particular measurement. The algorithms necessary to present these renderings and dimensions will require mathematical formulations that take into account various factors, including, but not limited to, offset of the balloon from the actual aortic tissue, averaging of the diametric dimensions of the annulus, systolic/diastolic movement, etc. Once the image has been rendered, the user may then be able to save that data in a file structure such that it can become a record for future use. There will also be a provision to allow for printing of the image.

There may be need for taking an image and then repositioning the balloon (or basket) and taking another image. The reconstructed image will allow the physician to accurately interpret the diameter of the annulus, the position of the coronary ostium and/or other desired aortic features in such a fashion as to aid in TAVI placement.

For purposes of illustration, the balloon embodiment is described in FIG. 4, however the methodology applies to a basket or a basket/balloon device design. The balloon method includes advancing the catheter 76 into the body of a patient, by directing the catheter distal end percutaneously through an incision and along a blood vessel until the balloon 70 is located within the annulus. The balloon 70 is positioned directly into the aorta such that, prior to inflation, the distal portion of the balloon approximates the aortic annulus. The balloon 70 is inflated to approximate the interior of the aorta, including the aortic annulus, while allowing for blood flow to continue through the balloon. Proper location of the balloon is confirmed through use of radiopaque markers located on the balloon and use of fluoroscopy with contrast media to define the vasculature as is typical practice. The balloon 70 will obtain measurement data of the aortic anatomy by use of a low pressure conformal balloon. This balloon 70 will contain several elements 72 disposed within the wall of the balloon 70 such that these elements 72 will come in contact either directly, or indirectly, with the aortic tissue. These elements will contain features that allow them to report a position within the aorta to a central reference point and also to each of the other elements. This information is used to construct a two or three dimensional recreation of the aorta via software residing in a data collection device 80. This software will interpret the position of each individual element relative to a central reference point along with the position of each element relative to the other elements such that construction of a two or three dimensional aortic representation is possible. In another embodiment, each data element will be able to determine its own particular position relative to itself and data from all elements will be combined via a software algorithm to construct a two or three dimensional aortic representation if possible. Once the construct has been rendered, the software allows for the selection of various anatomical features, including, but not limited to, the aortic annulus, for measurement. The software renders a two or three dimensional representation of the aorta on a data collection screen and enables the user to take necessary measurements directly from this graphical representation to select the proper TAVI device. TAVI devices are typically available in 23 mm, 26 mm, 29 mm and 31 mm diameters.

II. Devices for Introduction of Devices into the Vasculature

FIG. 8A is a schematic of a sheath showing the external structure of the introducer sheath into which a catheter for delivery of a device is inserted.

An embodiment of a vascular sheath introducer 10 is shown in FIG. 8A. The sheath introducer 10 has a first or distal end 12, and a second or proximal end 14 and a body 16, in a central or longitudinal axis. In a preferred embodiment, the sheath introducer is designed to dimensionally accommodate typical TAVI devices requiring vessels in the size range of 18-23 French.

The first or distal end 12 of the sheath is shown in FIG. 8A as the sheath tip 12. In some embodiments, the tip is an atraumatic tip, which can be selectively altered to minimize the degree of potential trauma to the surrounding tissue/vessel structures, typically through the selection of a soft, elastic covering, or “bumpers” that are positioned distal to the tip to help keep it away from the vessel wall during passage. An atraumatic tip on the introducer device additionally provides for the protection of ancillary vessels so that the chance for unintended vessel damage is reduced/eliminated. Preferably, the tip can be visualized via fluoroscopy so that the physician can position the device as needed to selectively navigate to the desired anatomy.

Referring to FIG. 8B, the tip 12 may be fabricated to allow the physician to steer the device as needed to the desired anatomical location during insertion. Tip 20 is steerable and deployable using a guidewire inside. Tip 22 shows the tip deflected upward by the guidewire. Tip 24 shows the tip defected downward by the guidewire.

The second or proximal end 14 remains external to the entry site when the device is inserted into the body. This second or proximal end 14 contains the device entry point 30, a sealing element 32 and a luer lock feature 34 or similar means for securing a device such as a syringe or catheter to the end. An injection port 36 is located on the sealing element 32. Proximal to the sealing element 32 is an anchor mechanism 38. The anchor mechanism 38 provides for the device to be properly secured at the entry site by use of sutures, adhesive, tape, clips, staples or other restraining means so that the device remains in place as positioned throughout the procedure. The anchor mechanism 38 should allow the sheath 10 to be repositioned/replaced without the need to re-suture/re-adhere the external positioning element to the patient.

The sheath body 40 is located in a central or longitudinal axis. The body has a length and diameter and obturator/needle/vessel dilator design to allow proper fit between the target vessel and the device as well as to optimize the length of the device so that when the device is positioned within the target anatomy, the exposed length of device exiting the patient is optimized. The desired sheath will range from 5 french to 12 french, with a preferred size range of 5 french to 8 french. The length of the sheath will range from 1.5 inches to 3.0 inches, with a preferred length of 2.0 inches. The needle/obturator size will range from 16 gage to 21 gage, with a preferred gage of 18 gage such that it will accommodate a 0.035 inch diameter guidewire. In addition, the needle is adapted such that the same needle is used to deliver the markers 41 as well as to access the subclavian artery.

The sheath introducer device may be coated to provide the necessary degree of lubricity required to reduce friction during placement. Lubricants or lubricous coatings can be used on the exterior of the sheath. Alternatively at least the outer surface of the sheath can be formed of a lubricous material. Lubricants for coating medical devices are known in the art. For example, a hydrophilic substance additionally can be applied to the outer surface of the sheath to act as a lubricant to case insertion of the sheath into a patient. U.S. Pat. No. 4,642,267 to Creasy et al., describes hydrophilic polymer blends (a thermoplastic polyurethane and a hydrophilic poly (N vinyl lactam) such as polyvinylpyrrolidone) useful for coating catheters and other surfaces. Additional components such as crosslinking agents and wax dispersions can be added to the blend. U.S. Pat. No. 4,675,361 to Ward, Jr. relates to polymer systems useful for coating surfaces having blood and tissue contacting applications. A variety of urethane based coating compositions for medical applications are known in the art. U.S. Pat. No. 5,272,012 to Opolski describes a coating solution which contains a protective compound such as a urethane, a slip additive such as a siloxane, and optionally, a crosslinking agent for the protective compound such as a polyfunctional aziridine, coating the solution onto a surface of a medical apparatus and allowing the coating to set.

In order to minimize the likelihood of localized infection, an anti-microbial/antifungal coating can be applied on the exposed areas of the device to reduce the possibility of bacterial/viral introduction/growth on the exposed device elements. Infections associated with medical device represent a major health care problem. A significant percentage of these infections are related to bacterial colonization of infusion catheters. Although any infectious agent can infect medical implants, Staphylococci (S. aureus, S. epidermidis, S. pyogenes), Enterococci (E. coli), gram negative Aerobic cacilli, and Pseudomonas aeruginosa are common pathogens. The introducer devices described herein are preferably coated with an antimicrobial composition, on the exposed areas of the device so as to reduce the possibility of bacterial/viral introduction/growth on the exposed device elements. Antimicrobial compositions and methods for coating antimicrobial compositions onto medical devices are known in the art. U.S. Pat. No. 5,624,704 to Darouchie, et al., describes antimicrobial impregnated catheters and other medical implants and method for impregnating catheters and other medical implants with an antimicrobial agent. U.S. Pat. No. 5,709,672 to Illner describes silastic and polymer-based catheters with improved antimicrobial/antifungal Properties”). Other methods for coating devices with antimicrobial/antifungal compositions are described, for example, in U.S. Pat. No. 5,520,664, to Bricault., et al., U.S. Pat. No. 5,902,283 to Darouchie, et. al U.S. Pat. No. 6,361,526 to Solomon, et al., and U.S. Pat. No. 6,261,271 to Solomon, et al. and International Publication No. WO2009105484.

The location of the sheath tip is important, insofar as the sheath must extend through the patient's vasculature. Accurate transit through the vasculature is facilitated by real time visualization of the tip of the introducer sheath. Oftentimes, the catheter or sheath tip is tracked or located by electrophysiological guidance, fluoroscopy, or a combination of the two. For an introducer sheath to be fluoroscopically visible it must be more absorptive of x-rays than the surrounding tissue. Radiopaque materials at the tip of the introducer allow for its direct visualization. Methods for making medical devices with flexible radiopaque tips are known in the art and can be adapted for the introducer sheath described above. For example, U.S. Pat. No. 5,045,072 to Castillo, et al., describes catheter with a flexible, distal tip. The distal tip comprises a plastic formulation containing sufficient radiopaque agent to be substantially more radiopaque than portions of the catheter proximal to the tip. See also for example, U.S. Pat. No. 5,558,652 to Henke and U.S. Pat. No. 6,884,235 to MacGukin, et al.

In another embodiment, the sheath is designed to work with a resectoscope, laparoscope or similar surgical instrument, such that the resectoscope, laparoscope or similar surgical instrument can be used to visually guide the sheath to a desired luminal target. Examples of resectoscopes include, but are not limited to, Wolf and Olympus, with diameters of 4 mm to 7 mm. Laparoscopes or similar surgical instruments may include those instruments that are dimensionally or otherwise suitable for use in this manner.

### III. Marker Needle Dispenser

A. Marker Needle Devices

A device has been developed to help image the site of implantation and route thereto. This is referred to as a marker needle dispenser, where the markers are a solution such as a radiopaque solution or suspension, which can be injected at a site where a measurement is made. The marker may be constructed of materials currently in use to provide radiopaque signatures. These materials are well known in the art and can be metallic, chemical or a combination of both and are typically manufactured as an integral part of the device. This marker may also be constructed such that the needle may touch it without trapping the catheter in the artery. This marker device may be constructed such that it can work with a resectoscope, laparoscope or similar surgical instrument, to dispense markers based on visual anatomical landmarks.

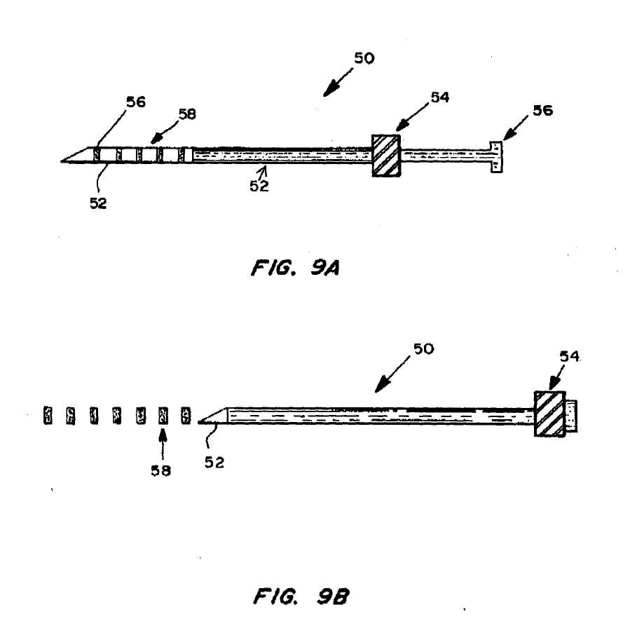

FIGS. 9A and 9B are schematics showing the marker needle dispenser. FIG. 9A shows the marker needle containing the market elements. FIG. 9B shows the deployed markers relative to the needle dispenser. The marker needle dispenser 50 is used to pre mark anatomical features that are either desirable to target, or are desirable to avoid, so that these elements can be utilized during the placement of the sheath. Referring to FIG. 9A, the marker needle dispenser 50 contains a needle 52, a needle carrier 54, and a marker dispensing plunger 56. The needle 52 contains a central passage or lumen for loading with marker material. FIG. 9A shows needle 52 loaded with marker elements 56, which are separated by spacing elements 58. The marker materials may be of any materials commonly used to provide radiopaque signatures in the human body. These materials include, but are not limited to, stainless steel, tantalum, nitinol, titanium, barium, and barium loaded polymers. The spacing between the markers may be accomplished by marker design, such that the marker defines the spaces between each marker. The spacing may also be accomplished by use of a non-radiopaque bioresorbable material that is left in situ during the procedure and disappears over time. Examples of these materials include, but are not limited to, alginate, catgut, polyglycolic acid (PGA), polylactic acid (PLA) and others similar biodegradable natural or synthetic polymers currently in use as resorbable materials. The desired length of spacing between markers can be controlled by the physician as deemed necessary during the procedure. Actual spacing of the markers is a physician preference and as such the device can be controlled in such a fashion as to allow the physician to place markers as deemed necessary.

FIG. 9B shows an empty marker needle 52, and deployed markers 56. The deployed markers enable the physician steer the sheath to the target site by following the markers. This also serves to identify areas the physician may desire to avoid.

The needle 52 shown in FIG. 9B has an elongated tubular shape having a constant-diameter inner bore and a constant-diameter exterior surface. The needle body can have other bore and exterior shapes (such as, for example, but without limitation, an oval cross-sectional shape). In a preferred embodiment, the needle has a length between 1.5 inches and 2.5 inches, most preferred, between 2.0 inches and 2.25 inches The size of the needle is between 16 and 21 gauge, a more preferably between 17 and 19 gauge.

The distal marker of the catheter based hemostasis system may be present in various configurations, but a preferred embodiment would be a round marker that would serve as a target for placement of the TAVI sheath.

B. Methods of Using Marker Needles

In one embodiment, anatomical features are pre-marked by delivering a marker or markers (via needle or other minimally invasive method) to the vessel entry site and inserted through the sheath, which may include a marker in the tip so that the sheath can be guided to the entry site. One can also dispense multiple markers by inserting a needle to locate the desired site and then dispensing markers in a predetermined sequence as the needle is removed. The physician is then able to steer the sheath to the target site by following the markers, or avoid marked sites.

In some embodiments, markers are used to guide entry and transition of the sheath at the entry site. The marker needle is used to provide visual location of anatomical features the physician wishes to avoid or approach, depending on the particular case. In one embodiment, the needle marker dispenser is used to mark the location of the desired entry into the subclavian artery such that the hemostasis device can be advanced to that particular site.

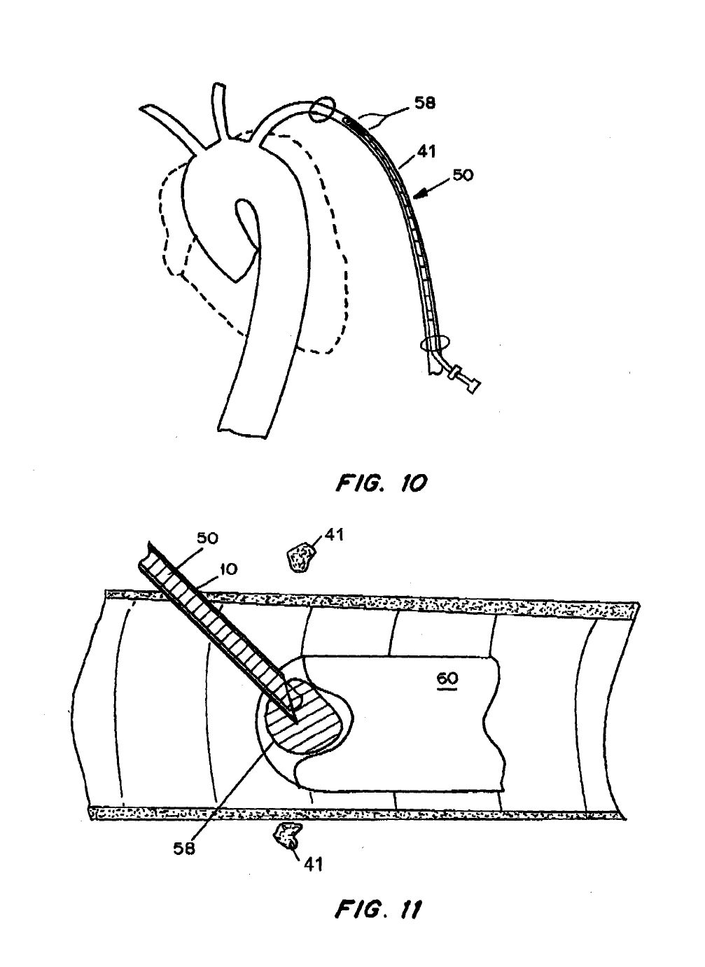

FIG. 10 is a prospective schematic showing the insertion of a marker needle into an artery such as the subclavian artery 62 and deployment of the markers to allow for sizing and location of insertion site dimensions.

Access is gained to the left radial artery 64 via typical Seldinger technique. Note: left radial artery diameters are slightly larger than right radial artery diameters, 23 mm versus 2.5 mm, respectively. An appropriate sheath introducer is placed in the radial entry site and advanced through the radial sheath introducer and positioned retrograde in the desired portion of the subclavian artery. In one embodiment, the distal most portion of the system carries a radiopaque marker. This marker is intended to define the preferred area of entry into the subclavian artery by the device via radiopaque signature. An additional marker(s) may be present to define the position of the device on the delivery catheter as well as to further aid in subsequent post TAVI deployment placement of the hemostasis device in the subclavian artery.

In another embodiment, the distal most portion of the system contains a light source that produces a full, or selective spectrum light such the light may be seen with the naked human eye, or in the case of selective spectrum, detectable by appropriate light detection devices. The light source is intended to define and identify the location of the preferred anatomical area of entry into the subclavian artery, in the example of use with TAVI procedures, by the device via visualization of the light signature.

### IV. Hemostatic Devices and Methods of Use Thereof

A. Hemostatic Devices

Devices have been developed to help insure closure at the site of introduction of the catheter and introducer sheath. This is particularly a problem with sites of entry such as into the sub-clavian artery, where it is difficult to immobilize and closure the artery.

The hemostatic devices generally fall into three categories: those that stabilize the site of entry, limiting movement of the blood vessel, providing support for the blood vessel and surrounding tissue to facilitate rapid closure by suture, staple or other means of closure; those that are moved into the site through another access point within the blood vessel so that it seals the opening even prior to closure but which can be removed after closure; and those that are left at the site to form a seal for a period of time as needed.

FIG. 11 is a cross-sectional schematic of a needle marker catheter 50 and introducer sheath 10 inserted into a hemostatic device 60 positioned adjacent to marker 58 inserted using the marker needle 50. This device stabilizes the point of entry and can deliver a self-sealing material that closes the entry site once the TAVI procedure has been completed.

Hemostatic device 60 introduced into the subclavian artery via the brachial artery assists in the prevention of bleeding at the site of introduction of a catheter and introducer sheath into the subclavian artery. Device 60 may be any gent type material that is used to seal a blood vessel or lumen. This may be degradable but is preferably formed of a non-degradable biocompatible material that is positioned to seal the site of entry immediately after removal of the catheter. This may be an expandable sleeve or mesh type stent. Alternatively, device 60 may be an angioplasty balloon that is expanded at the site to seal the entry point until hemostasis is obtained.

The distal marker 56 of the hemostasis system may be present in various configurations, but a preferred embodiment would be a round marker that would serve as a target for placement of the TAVI sheath.

In another embodiment, the device would include a light emitting distal marker as seen in FIG. 9A. This light emitting element may emit full or selective spectrum light energy such that the light is visible with the naked eye, or detectable with an instrument designed to detect specific wavelengths of light, through a luminal wall, such as a blood vessel.

B. Methods of Using Hemostatic Devices

In some embodiments, the hemostasis system (catheter, device and protective sheath), is advanced through the radial sheath introducer and positioned retrograde in the desired portion of the entry site for implantation of a device, for example, within the subclavian artery. In one embodiment, the device is introduced via another blood vessel that opens into the vessel in which the introducer sheath and device have been inserted, such as the brachial artery which enters the subclavical artery. The hemostasis device is moved into position immediately adjacent to the entry site prior to entry of the sheath and catheter used to place the implant. Then, as soon as the sheath and catheter are removed following the procedure, the hemostasis device is moved into position, sealing the opening. In the case of a stent or balloon, the hemostasis device is expanded in situ. It may be removed, as in the case of a balloon, once hemostasis has occurred, or it may be left in place, as in the case of a stent.

In other embodiments, the hemostasis device is positioned in situ, used to stabilize and seal the opening at the time of entry of the introducer sheath and catheter, then may be removed following removal of the sheath and catheter.

In one embodiment, the distal most portion of the hemostasis system carries a radiopaque marker. This marker is intended to define the preferred area of entry into the subclavian artery by the device via radiopaque signature. An additional marker(s) may be present to define the position of the hemostasis device on the delivery catheter as well as to further aid in subsequent post TAVI deployment placement of the hemostasis device in the subclavian artery.

The hemostasis device permits a Seldinger or a modified Seldinger technique to be used in gaining access to the subclavian artery for TAVI. In the Seldinger technique, the hemostasis device marker is placed in the desired vascular position for subclavian access, the radiopaque embodiment is imaged under fluoroscopy and the needle is inserted just below the left clavical. Under fluoroscopy, the needle is advanced until it touches the hemostasis device marker. Once entry into the vessel is confirmed, the hemostasis device is retracted until it is well out of the subclavian entry area. The TAVI sheath is positioned per typical sheath placement methodology.

A modified version of the Seldinger technique for placement into the subclavian artery would involve the same placement of the hemostasis. The difference would be that instead of using the standard needle, a marker needle would be used. This marker needle would be placed into the body. The marker needle is positioned above the hemostasis device marker, but not into the subclavian artery, and a marker dispensed. The same process is performed below the hemostasis device marker such that a target is radiopaqely defined (additional markers may be placed as desired). The marker needle would be removed and the hemostasis device would be retracted antegrade until out of the subclavian entry area. An access needle would then be inserted and guided, via fluoroscopy between the markers and into the target artery. Once entry into the vessel is confirmed, typical Seldinger technique is followed to place the sheath. Upon completion of these steps, placement of the TAVI device would then be performed as per normal procedure using the TAVI sheath introducer.

Once the TAVI procedure is finished, the TAVI sheath introducer is removed and the hemostasis device retrogradely positioned so that the vessel wound site is covered. If markers were used to gain access to the subclavian artery, then the hemostasis device is positioned such that the device provides sufficient coverage of the wound in reference to the markers. Successful placement of the hemostasis device is confirmed. Upon removal of the hemostasis device, the wound would be closed per standard practice and the procedure would be complete.

1. A balloon or basket catheter to determine two or three dimensional dimensions of a luminal region comprising
a balloon or basket having multiple elements thereon for sensing contact and transmitting signals to data collection or processing means to general an image reconstructed from the measurements obtained from the sensors,
catheter or guide wire means for intravascular delivery of the balloon or basket to a site to be measured, and means inflating or expanding the balloon or basket elements intraluminally.

2. The balloon catheter of claim 1 which allows for blood flow through the balloon during inflation.

3. The basket catheter of claim 1 comprising a catheter based, moveable basket design comprising at least two expandable elements which can be contacted at different points within a lumen, transmit a signal providing the degree of expansion, and contract for delivery and removal.

4. The basket catheter of claim 1, comprising an internal basket valve mechanism which, during the measuring process, can close to prevent flow or open to allow flow.

5. The basket catheter of claim 1 comprising an integrated dilatation balloon.

6. The basket catheter of claim 1 further comprising wireless or hard wired data transmission means.

7. A sheath introducer comprising
A hollow lumen having a steerable, deployable atraumatic entry tip at a first end, which optionally is radiopaque or fluoroscopically visualizable, and
a sealing element at the end distal to the entry tip, containing a device entry point.

8. The sheath introducer of claim 7 comprising a luer lock feature at the end distal to the entry tip.

9. The sheath introducer of claim 7 comprising an introduction port at the end distal to the entry tip.

10. The sheath introducer of claim 7 wherein an oscope, laparoscope or other similar surgical instrument may be used in conjunction with the sheath to aid in placement within the target anatomy.

11. A marker needle dispenser comprising a needle and markers in a needle carrier having means for deploying the markers, the markers having specific spacing, coloring, shape, or imaging characteristics that can be used to distinguish the deployed markers, and the needle containing a central passage or lumen for loading marker and spacer material.

12. The marker needle of claim 11 in a kit or in conjunction with a resectoscope, laparoscope or other similar surgical instrument.

13. A lumen sealing device comprising
A catheter based stent or balloon device capable of expanding to seal a lumen such as a blood vessel, the stent or balloon comprising means for allowing introduction of a catheter or sheath.

14. The catheter based stent or balloon device of claim 13 that contains a full or partial spectrum light emitting element that is viewable when used with a resectoscope, laparoscope or similar surgical instrument.

15. The catheter based stent or balloon device of claim 13 that contains a single or series of selectively placed radiopaque markers for use in properly positioning the catheter within the target anatomy when used with fluoroscopy.

16. A method of decreasing fluid flow, such as bleeding, or facilitating closure of a catheter, sheath or device entry site into a lumen such as a blood vessel, comprising providing at the site immediately after removal of the catheter, sheath or device an expandable device applying pressure to the entry site and stabilizing fluid flow at the lumen site.

17. The method of claim 16 wherein the expandable device is a stent and/or balloon.

18. The method of claim 16 wherein the expandable device is removed after fluid flow, such as bleeding, through the entry site is stopped, through a different site.

19. The method of claim 16 wherein the expandable device is left in place as a permanent implant.

20. The method of claim 18 wherein the expandable device is positioned in the lumen located immediately adjacent the entry site, prior to entry of the catheter, sheath or device.

21. The method of claim 16 wherein the expandable device contains selectively positioned radiopaque markers such that the device can be accurately positioned within the anatomy to facilitate proper location at the entry site.

22. A method of use of the device of claim 1.

23. A method of use of the device of claim 7.

24. A method of use of the device of claim 11.

25. A method of use of the device of claim 13.
